(19) United States
(12) Patent Application Publication
(10) Pub. No.: US 2021/0128196 A1
(43) Pub. Date: May 6, 2021

(54) MODULAR TISSUE RETRACTOR DEVICES, SYSTEMS, AND METHODS OF USE

(71) Applicant: Duke University, Durham, NC (US)

(72) Inventors: Kathleen Carroll, Durham, NC (US); Luis Fernando Gonzalez, Durham, NC (US); Aimee Mcvey, Durham, NC (US); Anthony Haddad, Durham, NC (US); Charles Geddie, Durham, NC (US)

(21) Appl. No.: 17/091,409

(22) Filed: Nov. 6, 2020

Related U.S. Application Data
(60) Provisional application No. 62/931,390, filed on Nov. 6, 2019.

(51) Int. Cl. A61B 17/34

(52) U.S. Cl. CPC A61B 17/3423; A61B 17/3415; A61B 2017/3443; A61B 2017/3492; A61B 2017/347

(57) ABSTRACT

The present disclosure provides devices, systems, and methods relating to performing a medical procedure. In particular, the present disclosure is directed to modular tissue retractor devices that include a cannula with modular and stackable axial segments, an accompanying obturator, and an adjustable mounting device. The devices and systems described herein facilitate tissue retraction prior to and/or during a medical procedure without removing the cannula from a target tissue.

### RELATED APPLICATIONS

This application claims priority to and the benefit of U.S. Provisional Patent Application No. 62/931,390 filed Nov. 6, 2019, which is incorporated herein by reference in its entirety for all purposes.

### FIELD

This present disclosure provides devices, systems, and methods relating to performing a medical procedure. In particular, the present disclosure is directed to modular tissue retractor devices that include a cannula with modular and stackable axial segments, an accompanying obturator, and an adjustable mounting device. The devices and systems described herein facilitate tissue retraction prior to and/or during a medical procedure without removing the cannula from a target tissue.

### BACKGROUND

Access to an injury site is critical for all medical procedures (e.g., surgical interventions). One must be able to physically manipulate the human body to correct abnormalities while minimizing damage to the healthy tissue. This ideal is most important to procedures that seek to heal sensitive tissues in our brain and spinal column. However, currently tissue retracting technology for the spine and brain either incorporates hard edge implements that cause tissue damage or insertable devices that often need replacement in the middle of the surgery due to changing geometric conditions, at times also causing tissue damage upon removal. The unique nature of each patient and each surgical intervention requires that highly specialized, often disposable devices have been made, increasing the overall cost of the procedure without addressing patient outcomes. Therefore, development of a device that reduces tissue damage upon insertion and can be adjusted in situ has the potential to unilaterally improve patient outcomes for a variety of neurological procedures.

To assist in visualization during intracranial procedures, practitioners often move aside brain tissue to expose the region of interest, a process referred to as retraction. Brain retraction is particularly useful when accessing deep-seated lesions. Several types of tissue retractors are available. Traditional flat blade retractor systems can often be bulky and cumbersome to use and have been associated with cortical and vascular damage. Tubular retractors are also available, which equalize pressure distribution on the surrounding brain and thus limit the transection of white matter tracts while maintaining lesion access. These devices are typically available in fixed port diameters and lengths, selected pre-operatively based on CT or MRI brain imaging. However, once surgery begins, surgeons often find the need to access greater depths or modify angles intra-operatively to completely visualize and dissect the addressed lesion. Currently, to adjust retractor length, surgeons must completely remove the device and insert a new retractor, as in some cases, the device will protrude outwardly as the procedure is performed (e.g., deep to shallow), which interferes with the ability of surgeons to manipulate their instruments. This not only increases cost of procedure but also leads to added trauma to the surrounding brain. Hence, there is an ongoing need for improvements to tubular brain retractors.

### SUMMARY

Embodiments of the present disclosure provide a tissue retraction system that includes an obturator comprising a housing for one or more surgical tools, and a cannula configured to accept the obturator and comprising a plurality of axial segments. In accordance with these embodiments, the plurality of axial segments include at least one distal base segment and at least one proximal top segment. In some embodiments, the plurality of axial segments are modularly stackable and configured to be added and/or removed prior to and/or during a medical procedure without removing the cannula from the tissue.

In some embodiments, the plurality of axial segments of the cannula comprises one or more extension segments disposed between the distal base segment and the proximal top segment.

In some embodiments, the proximal top segment comprises an extension tab for interfacing with an external retractor tool.

In some embodiments, the surgical tools that can be used with the tissue retractor systems include, but are not limited to, one or more of an aspiration canula, a micro-dissector, a micro-scissors, a bipolar electrocoagulation image guidance probe, an ablation tool, a tissue removal tool, or a combination thereof.

In some embodiments, the obturator comprises a retention element to secure the one or more surgical tools in position.

In some embodiments, at least one area along the longitudinal axis of the obturator is planar and interfaces with at least one corresponding area along the longitudinal axis of the cannula to prevent rotation of the obturator within the cannula.

In some embodiments, the distal base segment of the cannula comprises a plurality of splines on its inner surface that interface with a corresponding plurality of splines on the outer surface of the obturator to prevent rotation of the obturator within the cannula.

In some embodiments, the plurality of axial segments of the cannula comprise an external locking feature and/or an internal locking feature, such that each segment can be removably coupled to a separate segment. In some embodiments, the plurality of axial segments of the cannula are from about 5 mm to about 50 mm in height. In some embodiments, the plurality of axial segments of the cannula are from about 5 mm to about 50 mm in diameter.

In some embodiments, the obturator comprises two proximally positioned buttons functionally coupled to two distally located flanges, such that depressing the buttons causes the flanges to engage the cannula, thereby facilitating removal of the cannula upon removal of the obturator.

In some embodiments, the proximal end of the obturator comprises an external locking feature or an internal locking feature that engages a corresponding internal or external locking feature in the proximal end of the cannula, such that engagement of the proximal end of the obturator with the proximal end of the cannula facilitates removal of the cannula using the obturator.

In some embodiments, the tissue accessed using the systems described herein is neural tissue (e.g., brain tissue).

In some embodiments, the system further comprises an adjustable mount configured to position the cannula in one or more stable positions.

In some embodiments, the adjustable mount comprises a base for securing the mount under the scalp of a subject, an adjustable cannula attachment portion comprising an opening for insertion of the cannula, wherein at least a portion of the adjustable cannula attachment portion is recessed in the base, and a tension cap configured to apply varied amounts of pressure to the cannula attachment portion to secure the cannula in one or more stable positions.

In some embodiments, the base comprises at least two opposing base tabs and/or a concentric lip at the bottom of the mount.

In some embodiments, the adjustable cannula attachment portion has a concave shape with respect to the tension cap.

Embodiments of the present disclosure also include an adjustable mounting device for at least one medical tool. In accordance with these embodiments, the device comprises a base for securing the mount to a subject, an adjustable medical tool attachment portion comprising an opening for insertion of at least one medical tool, wherein at least a portion of the adjustable medical tool attachment portion is recessed in the base, and a tension cap configured to apply varied amounts of pressure to the medical tool attachment portion to secure the tool in one or more stable positions.

In some embodiments, the base comprises at least two opposing base tabs and/or a concentric lip at the bottom of the mount.

In some embodiments, the base tabs and/or the concentric lip facilitate attachment to the subject without the need for additional attachment means.

In some embodiments, the base tabs are perforated for insertion of an additional attachment means to the subject.

In some embodiments, the adjustable medical tool attachment portion has a concave shape with respect to the tension cap.

In some embodiments, the at least one medical tool comprises a cannula, bipolar forceps, dissectors, biopsy forceps, a suction device, a camera light source, a guidance probe, coagulation forceps, micro-instruments, and the like.

In some embodiments, the base secures the mount to the subject's skull.

In some embodiments, securing the medical tool in one or more stable positions comprises securing the tool at an angle with reference to a surface-to-target location. In some embodiments, the angle is about 20 degrees or less.

Embodiments of the present disclosure also include a method of performing a medical procedure using the systems described herein. In accordance with these embodiments, the method comprises inserting the cannula and obturator into the tissue adjacent to a target lesion using an guidance probe, and adjusting the length of the cannula by adding or removing at least one axial segment.

In some embodiments, the method is performed prior to and/or during a surgical procedure, wherein the cannula is not fully removed until completion of the surgical procedure.

In some embodiments, inserting the cannula and obturator comprises use of an adjustable cannula mounting device, wherein the cannula and obturator are inserted at an angle with reference to a direct target lesion-to-scalp axis.

### BRIEF DESCRIPTION OF THE DRAWINGS

FIGS. 1A-1C include representative views of tissue retraction devices and systems of the present disclosure. FIG. 1A is a cross-sectional view of a tissue retractor system, and FIGS. 1B-1C are elevation views of the embodiment of FIG. 1A, according to one embodiment of the present disclosure.

FIGS. 2A-2C includes a cross-sectional view of the embodiment of FIG. 1A illustrating one embodiment for preventing rotational movement of the obturator within the cannula (FIG. 2A), and a longitudinal view of a separate embodiment that includes a splined interface between the obturator and cannula for preventing rotational movement of the obturator within the cannula (FIG. 2B-2C).

FIG. 3 is a detailed view of the embodiment of FIG. 1A.

FIG. 4 is a perspective view of a retractor system that includes a cannula comprising a distal base segment and a proximal top segment, according to one embodiment of the present disclosure.

FIG. 5 is a perspective view of a retractor system that includes a cannula comprising a distal base segment, a proximal top segment, and additional extension segments, according to one embodiment of the present disclosure.

FIG. 6 is a perspective view of a retractor system that includes a cannula comprising a distal base segment, a proximal top segment, and an additional extension segment, according to one embodiment of the present disclosure.

FIGS. 7A-7D include representative views of a retractor system comprising extension tabs for interfacing with an external retractor tool (FIG. 7C), including various views of proximal top segments comprising extensions tabs (FIGS. 7A-7B), according to one embodiment of the present disclosure. FIG. 7D includes representative views of a retractor system comprising an internal locking feature as part of the proximal end of the obturator for interfacing with a corresponding external locking feature in the proximal end of the cannula to facilitate removal of the cannula using the obturator, as shown (FIG. 7D).

FIG. 8 includes representative views of various proximal top segments, including an embodiment with an angled proximal surface, according to one embodiment of the present disclosure.

FIG. 9. includes representative views of various proximal top segments, including an embodiment with a horizontal proximal surface, according to one embodiment of the present disclosure.

FIGS. 10A-10B include representative views of various axial segments, including embodiments comprising internal and external locking features, according to one embodiment of the present disclosure.

FIGS. 11A-11B include representative views of various distal base segments, including embodiments comprising internal and external locking features, according to one embodiment of the present disclosure.

FIG. 12 includes representative illustrations of a method of joining axial segments of a cannula, according to one embodiment of the present disclosure.

FIGS. 13A-13C include various cross-sectional views of a retractor system (FIG. 13A) comprising an obturator (FIG. 13B) and a cannula (FIG. 13C), according to one embodiment of the present disclosure.

FIGS. 14A-14E include various views of an obturator, including embodiments comprising two proximally positioned buttons functionally coupled to two distally located flanges, according to one embodiment of the present disclosure.

FIGS. 15A-15B include various views of a tissue retraction system comprising an adjustable mounting base, according to one embodiment of the present disclosure.

FIG. 16 includes representative images of an adjustable mount for insertion of a tissue extraction system held in a stable position by skin tension fixation (left panel) and/or fixation screws (right panel), according to one embodiment of the present disclosure.

FIG. 17 includes representative views of a retraction system comprising an adjustable mount, according to one embodiment of the present disclosure.

FIG. 18 includes representative views of a retraction system comprising an adjustable mount, according to one embodiment of the present disclosure.

FIG. 19 includes representative views of a retraction system comprising an adjustable mount, according to one embodiment of the present disclosure.

FIG. 20 includes representative views of an adjustable mount for a tissue retraction device or system that allows for multiple different angles of operation, according to one embodiment of the present disclosure.

FIG. 21 includes representative views of a tissue retraction system comprising a cannula, obturator, and adjustable mount, in which the cannula is positioned to be directly above a target lesion (middle and right panels) or rotated at an angle with reference to a target lesion (left panel).

FIG. 22 includes representative views of a tissue retraction system comprising a cannula (open channel shown) and adjustable mount, in which the cannula is positioned to be directly above a target lesion (middle and right panels) or rotated at an angle with reference to a target lesion (left panel).

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the treatment comprises the treatment, alleviation, and/or lessening of pain.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Tissue Retraction Devices and Systems

Embodiments of the present disclosure relate generally to tubular tissue retractors. Tubular retractors are known in the art to be the retractor-of-choice for procedures that are between open surgery and minimally invasive surgery; for example, they can be especially useful for intracerebral hematoma evacuation because they provide quick access to a target lesion. Tubular retractors are also useful when both visualization and treatment of a target lesion requires multiple medical tools/instruments. Tubular retractors can also be used to resect tumors (e.g., meningioma, or solid tumors), as a tube allows a surgeon to resect one piece at a time, or to evacuate a liquid mass (e.g., hematoma or cyst). Tubular retractors can also be used as a suclcus dilator, where the surgeon creates a path and then removes the tube to operate.

As described further herein, embodiments of the present disclosure provide an improved tubular retractor device/system. In particular, the present disclosure is directed to modular tissue retractor devices that include a cannula with modular and stackable axial segments, an accompanying obturator, and an adjustable mounting device. The devices and systems described herein facilitate tissue retraction, while minimizing pressure along a tract, prior to and/or during a medical procedure without removing the cannula from a target tissue. Target tissue can include, but is not limited to, neural tissue, cerebral ventricles, cerebral parenchyma, the spine, and other solid organs such as liver, spleen or kidney, and the like. As described further herein, the devices and systems of the present disclosure can be used to treat any lesion in a target tissue, including but not limited to, a cystic lesion, an infection (e.g., cerebral abscess), a primary or metastatic tumor, an intracerebral or intraventricular hemorrhage, an intracranial hematoma, a vascular abnormality or malformation, an intraventricular cyst, and the like.

For example, prior to performing a medical procedure, a surgeon plans the trajectory to estimate the length of cannula. It is generally advantageous to choose the shortest cannula possible that will access the abnormality to maximize visualization and usability of tools. However, the mound of muscle/skin made from a skin incision may affect depth measurements. In this instance, the surgeon would have to remove the device and insert a longer one, which increases costs, procedure time, and can interfere with the ability of the surgeon to perform the procedure safely and efficiently. The embodiments of the devices/systems of the present disclosure enable the surgeon to simply add/remove an additional cannula component to provide the extra needed length, which reduces cost and enhances functionality. Similarly, when a procedure begins at the most distal aspect of a lesion, and then advances, the retractor will extend outwardly, making it difficult and cumbersome for the surgeon to manipulate many micro-instruments. The embodiments of the present disclosure enable a surgeon to remove the most proximal segments, thereby providing a shorter, cleaner operating field.

Additionally, the tissue retraction devices and systems of the present disclosure include a reduced profile at the surface of a subject's body. Current products have an offset to interface with, for example, a Shepard's hook attachment, preventing tangential placement to the skull. Additionally, bunched up skin/fat/muscle at the site of the incision may increase the depth. The longer the tube, the more difficult it is to manipulate during a medical procedure, which constrains mobility. The devices and systems of the present disclosure have a low-profile (e.g., little to no rim or lip around the top of the cannula) design for placement completely into a burr hole, if desired.

Embodiments of the present disclosure also allow for multi-axial positioning for precise access to a target lesions. That is, use of a Shephard's hook is bulky and does not often provide the stable positioning of the cannula that is required for a medical procedure (e.g., without the need for additional personnel to manually hold the cannula throughout a procedure). In contrast, the devices and systems of the present disclosure do not require connection to an external table-fixed retractor system, for example. The systems of the present disclosure are independent to a table fixed retractor system, thereby negating retractor arms that are often cumbersome and reduce efficiency of a procedure. As described further herein the use of an adjustable fixation mount allows the user to adjust the trajectory angle in multiple planes (e.g., to access the target lesion from multiple angles). The use of adjustable locking mechanisms also minimize unintended cannula movement during a procedure. That is, the user may firmly lock the position by twisting the top part of the mount, or may simply increase the resistance, so that they can adjust the position of the tube in the tissue while preventing unintentional movements caused by hysteresis of the brain. Additionally, repositioning can be performed using the obturator and/or the medical tools within the cannula.

These and other advantages would be readily apparent to one of ordinary skill in the art based on the present disclosure.

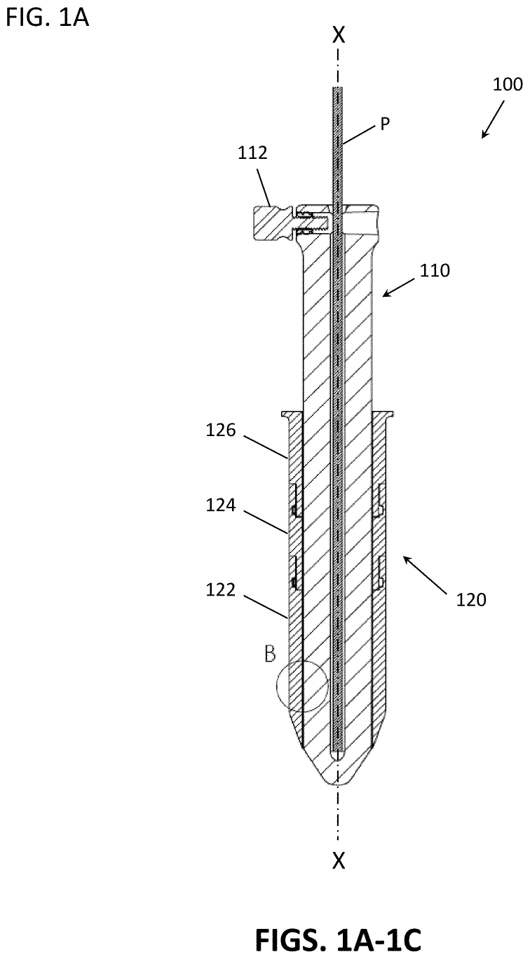

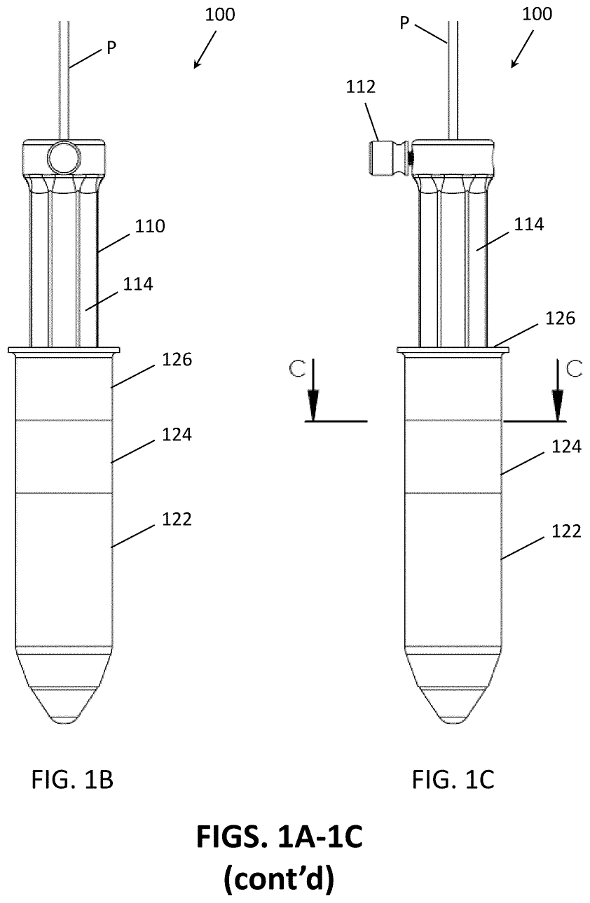

Turning to the embodiments represented in the figures, the tissue retraction devices and systems of the present disclosure include use as a brain tissue retraction device, generally designated 100 (FIGS. 1A-1C). FIG. 1A is a cross-sectional view of a tissue retractor system, and FIGS. 1B-1C are elevation views of the embodiment of FIG. 1A, according to one embodiment of the present disclosure. As illustrated in the cross-sectional assembly in FIG. 1A, retractor system 100 has two main components: an obturator 110 and a cannula 120. Obturator 110 is configured to include a housing for one or more surgical tools, such as but not limited to, a guidance probe P (e.g., image guidance probe or ultrasound image guidance probe). Other surgical tools include, but are not limited to, one or more of an aspiration canula, a micro-dissector, a micro-scissors, a bipolar electrocoagulation forceps, an image guidance probe, an ablation tool, a tissue removal tool, or a combination thereof. As shown in FIGS. 1A-1C, obturator 110 can also include a retention element 112 to secure the one or more surgical tools in position.

In some embodiments, the obturator 110 is inserted within cannula 120. Probe P can be secured in place in obturator 110 via a retention element 112. The assembled retractor system 100 can be used to, for example, dilate cerebral sulci and assist in proper placement of cannula 120 inside the brain of a subject, whereupon obturator 110 and probe P can be removed from cannula 120, and visualization of the cerebral region of interest (e.g., a lesion) is possible.

The length of cannula 120 can be adjusted using a plurality of stackable axial segments. In the example embodiment of FIG. 1, cannula 120 includes a distal or base segment 122, one extension segment 124, and a proximal or top segment 126. As will be described in further detail below, these segments can be selected based on the requirements of the application for which retractor system 100 is used. That is, the plurality of axial segments are modularly stackable and configured to be added and/or removed prior to and/or during a medical procedure without removing the cannula from the target tissue. The segments can be arranged in series along a longitudinal axis X of cannula 120. Base segment 122 has features for positioning obturator 110, as well as a distal profile that is conducive to penetrating the brain tissue. In the example embodiment of FIG. 1, the distal base segment 122 has an open, beveled tip for receiving obturator 110. The proximal top segment 126 can have varying features adapted for manipulation or fixation of cannula 120. One or more extension segments 124 can be selected based on an estimated length necessary to reach the target tissue.

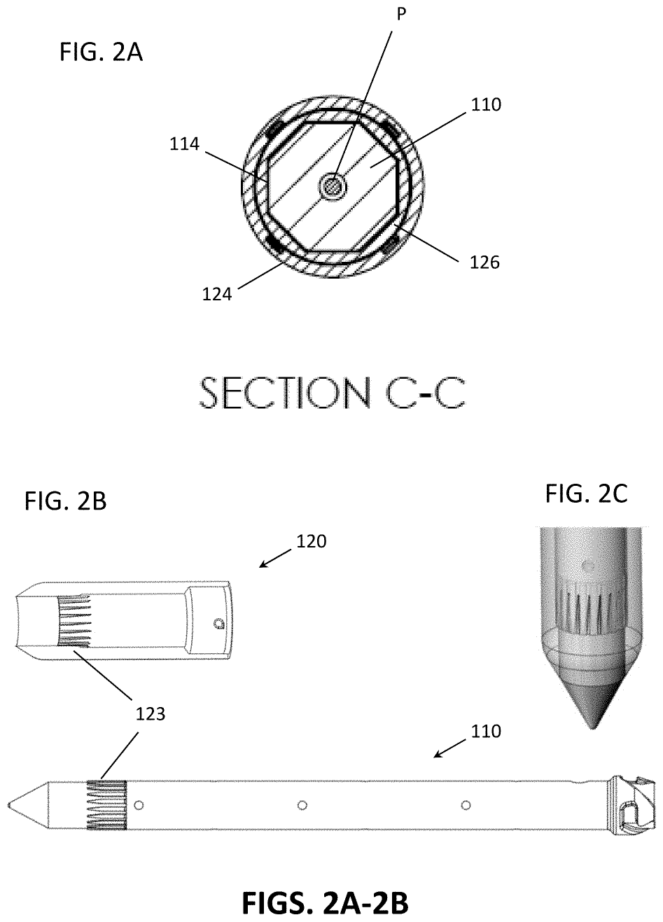

As shown in FIGS. 1B and 1C, obturator 110 has optional rotational locating features 114. That is, in some embodiments, at least one area along the longitudinal axis of the obturator 110 is planar and interfaces with at least one corresponding area along the longitudinal axis of the cannula 120 to prevent rotation of the obturator within the cannula. In the example embodiment of FIGS. 1A-1C, locating features 114 are in the form of flats or facets on an outer surface of obturator 110. Locating features 114 are used to prevent rotation of obturator 110 inside cannula 120. This is also shown in FIG. 2, which is a cross-sectional view of FIG. 1C taken through section C-C. Obturator 110 is substantially in contact with an inner surface of cannula 120, thus preventing relative rotation of the two parts and assisting the practitioner in navigation of retractor 100. Although rotational locating feature 114 is depicted as an octagon, it is possible for the feature that interlocks the obturator and the cannula to have any suitable shape for preventing rotation. For example, as shown in FIGS. 2B-2C, rotational locating features 114 can include a plurality of splines 123 on the inner surface of the cannula 120 that interface with a corresponding plurality of splines 123 on the outer surface of the obturator 110 to prevent rotation of the obturator within the cannula. Other examples include a circular shape with a flat or a key; other geometric profiles such as hexagons; and other conventional mating features such as pins, springs, bosses, etc. Further possibilities will also be evident to a person of ordinary skill in the art based on the present disclosure.

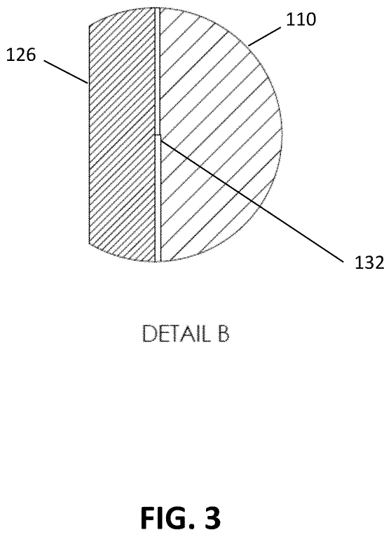

In addition to rotational locating feature 114, retractor system 100 also has a vertical locating feature or stop 132. This is shown in FIG. 3, which is an exploded view of section B in FIG. 1A. Stop 132 prevents obturator 110 from advancing past a predetermined depth relative to cannula 120. In this example embodiment, stop 132 is in the form of a lip or flange on the inner surface of base segment 122 and complementary mating shape on obturator 110. Similar to rotational locating feature 114, stop 132 can take any suitable form. In addition to preventing advancement and moving the obturator and the cannula together, stop 132 also ensures that obturator 110 always protrudes the same distance from cannula 120. This assists in skin and subcutaneous retraction and provides a smooth and continuous interface between the outside surfaces of obturator 110 and cannula 120. The known distance allows calibration of imaging probe P. This can help provide accurate navigation and reduce the risk of injury to the subject. In a non-limiting example, obturator 110 can extend approximately 5 mm from the distal opening of cannula 120. In some embodiments, the obturator 110 extends about 5 mm to about 10 mm from the distal opening of the cannula 120. In some embodiments, the obturator 110 extends about 5 mm to about 7 mm from the distal opening of the cannula 120.

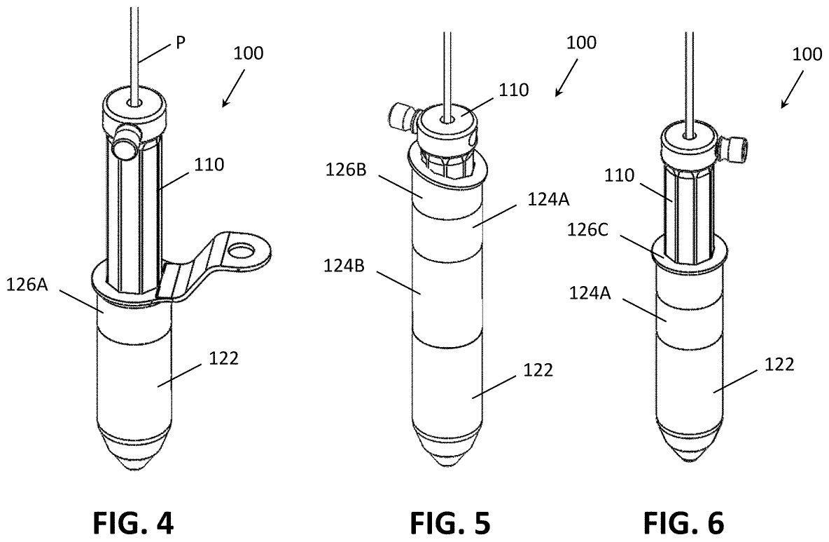

FIGS. 4-6 depict several possible example configurations for cannula 120. Because the cannula 120 is configured to comprise a plurality of axial segments (e.g., at least one distal base segment 122 and at least one proximal top segment 126), its height can be adjusted prior to and/or during a medical procedure without removing the cannula 120 itself. The plurality of axial segments are designed to be assembled in a modular fashion, so the length of cannula 120 can be adjusted to accommodate a variety of surgical applications. Cannula 120 is particularly adaptable when the application is exploratory or uncertain, because the tubes can be added or removed while the base section of the cannula remains in situ.

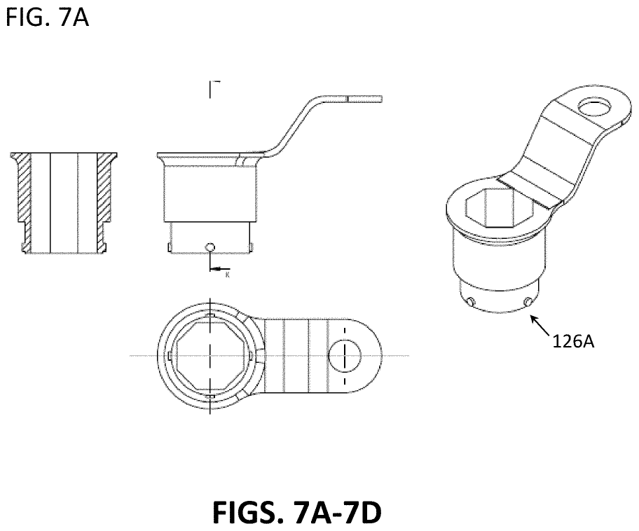

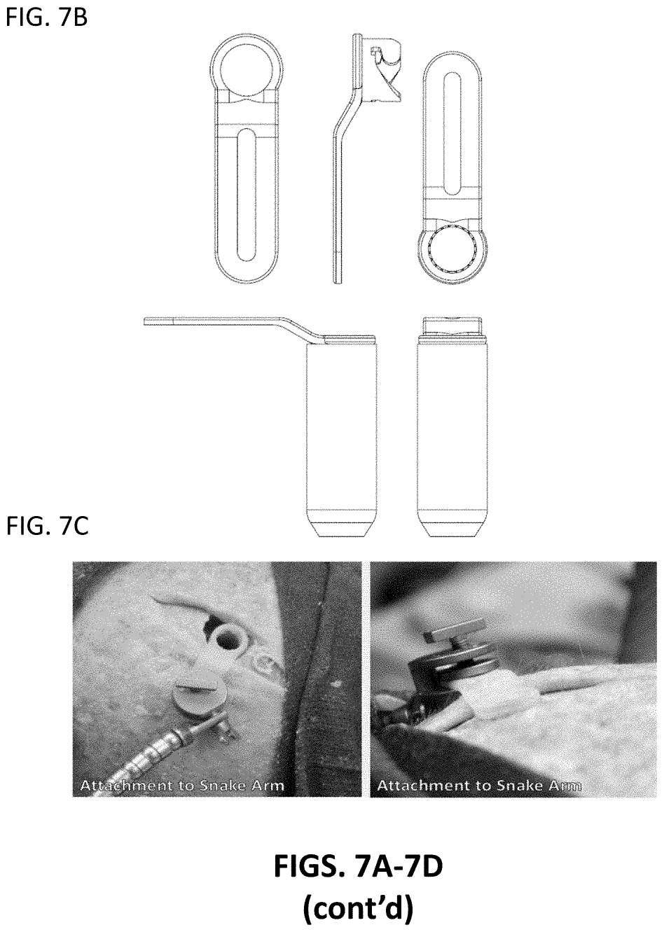

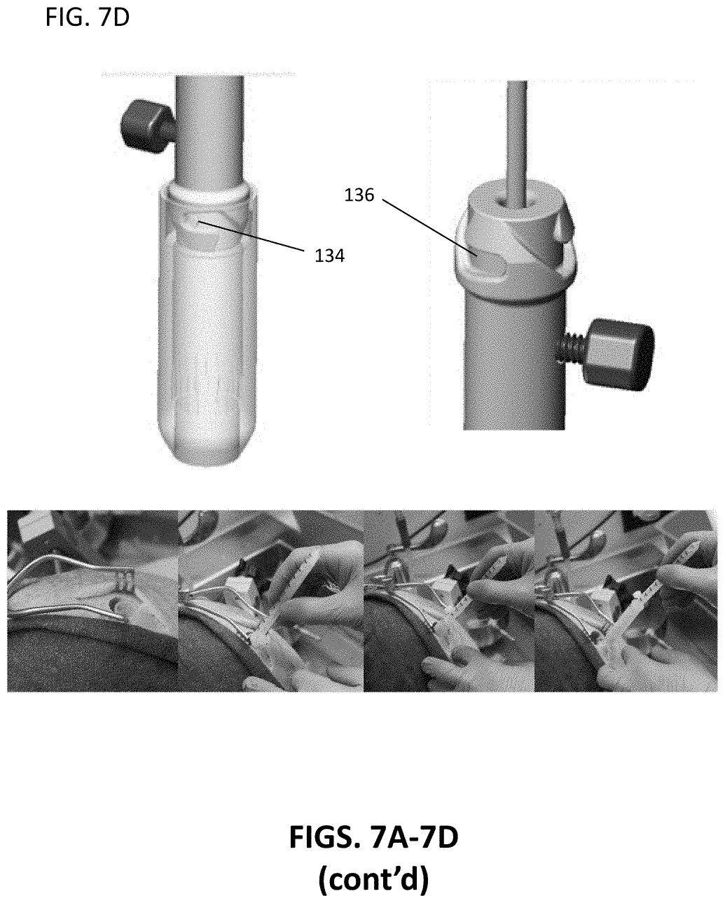

For example, FIG. 4 depicts an embodiment of retractor system 100 that uses only a base segment 122 and a top segment 126A. Top segment 126A includes an extension tab for interfacing with an external retractor tool (e.g., a “Shepherd's hook” style tab; see also FIG. 7), which can be manipulated by hand or connected to commercially available surgical equipment. Top segment 126A can be attached directly to base segment 122 through the mating features described in further detail below. Note, it is also evident from FIG. 4 that it is not necessary for obturator 110 to be the same length as cannula 120. Obturator 110 can be provided in a standard length that is longer than cannula 120 and extends from the proximal opening.

FIG. 5 depicts another embodiment of retractor system 100. In this embodiment, two extension segments 124A and 124B are assembled between base segment 122 and top segment 126B. Multiple extension segments 124 can be stacked together to achieve a prescribed overall length of cannula 110. Further, extension segments 124 can be provided in various lengths, such as in a kit of parts, in order to be added as needed during a medical/surgical procedure. In a non-limiting example, extension segments 124 can be from about 5 mm to about 50 mm in length. In some embodiments, extension segments 124 can be from about 5 mm, about 10 mm, about 15 mm, about 20 mm, about 25 mm, about 30 mm, about 35 mm, about 40 mm, about 45 mm, and about 50 mm in length.

In some embodiments, proximal top segment 126B, as shown in the example embodiment of FIG. 5, has an angled proximal surface. This can advantageously allow retractor system 100 to be inserted at an oblique angle (e.g., to the skull) without diminishing the working space above the insertion area. That is, the outer edge of cannula 120 can be oriented so that the angular surface is tangential to the skull, resulting in a larger cross-sectional opening that is accessible from a wider angle. Currently, many conventional cannulas cannot be placed flush with the skull, causing the surgeon to lose valuable cannula length and visibility. In some embodiments, the angle of the proximal surface can be any angle from horizontal, e.g., about 1° to about 45° from horizontal, where “horizontal” refers to a direction perpendicular to the longitudinal axis of the cannula. Other angular values are also possible, as would be recognized by one of ordinary skill in the art based on the present disclosure.

FIG. 6 depicts another example embodiment of retractor system 100. In this embodiment, base segment 122 is combined with one extension segment 124A and a top segment 126C. Top segment 126C has a simple horizontal proximal surface. In some embodiments, top segment 126 comprises little to no rim or lip around its most proximal portion (e.g., a low-profile design or “rimless”); in such embodiments, the cannula can be removed using the obturator itself rather than using an external retractor device, as described further below (see, e.g., FIG. 7D). Similarly to extension section 124, a variety of top segment 126 can optionally be provided in a kit of parts, in order for the practitioner to be able to change the length and the top tube configuration during a procedure.

FIGS. 7A-7D include representative views of a retractor system 100 comprising extension tabs for interfacing with an external retractor tool (FIG. 7C) for removing cannula 120, including various views of proximal top segments 126 comprising extensions tabs 124 (FIGS. 7A-7B). Additionally, FIG. 7D includes representative views of a retractor system comprising an internal locking feature 136 as part of the proximal end of the obturator for interfacing with a corresponding external locking feature 134 in the proximal end of the cannula 120 to facilitate removal of the cannula 120 using the obturator 110. As shown in the bottom images of FIG. 7D, cannula 120 can be removed using the obturator 110, which obviates the need for an additional extractor tool/mechanism.

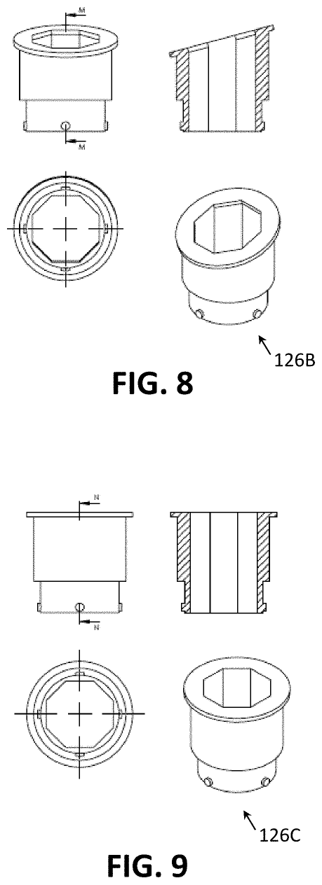

FIGS. 7-9 further illustrate detailed views of the example embodiments of 126A-126C, respectively. FIG. 7A includes various views of an embodiment 126A using an extended tab or Shepherd's hook style end. FIG. 8 is a top segment 126B with an angled proximal surface, and FIG. 9 is an embodiment 126C with a substantially perpendicular proximal surface. Each of these embodiments has a flange or lip surrounding the proximal opening.

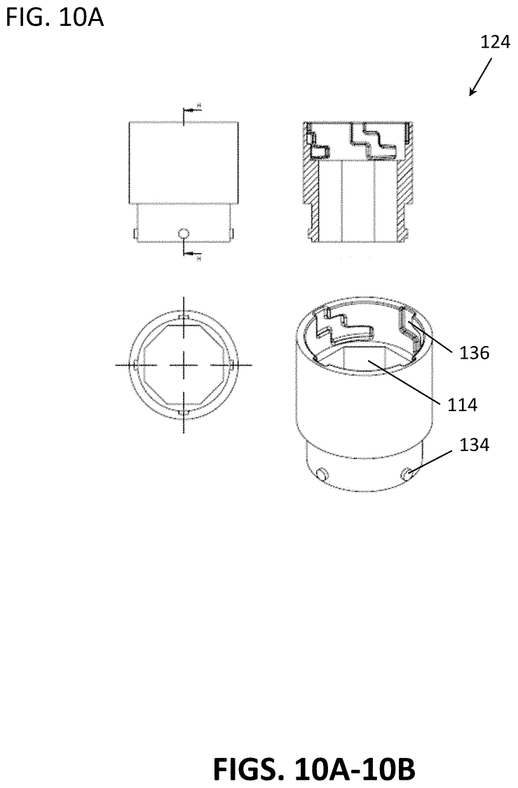

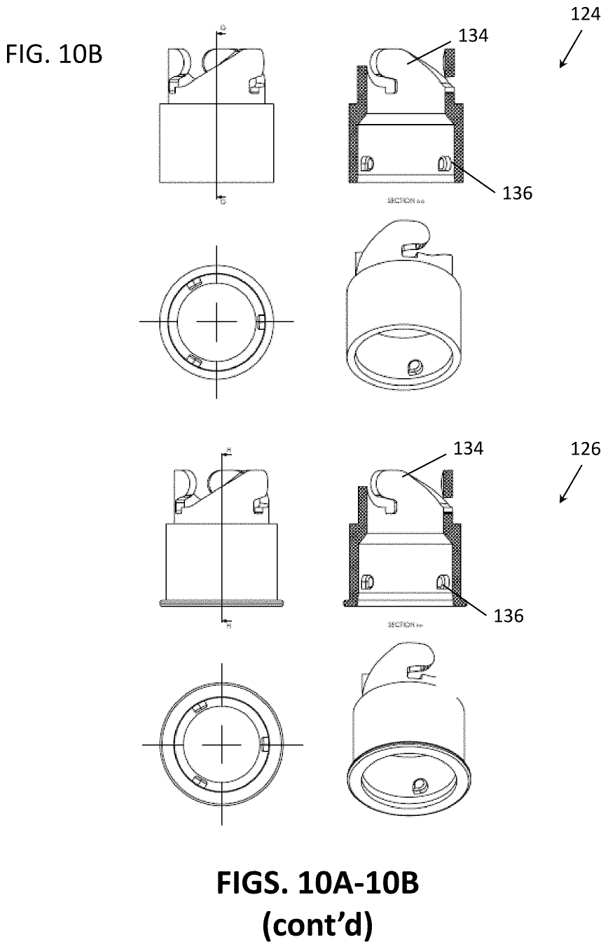

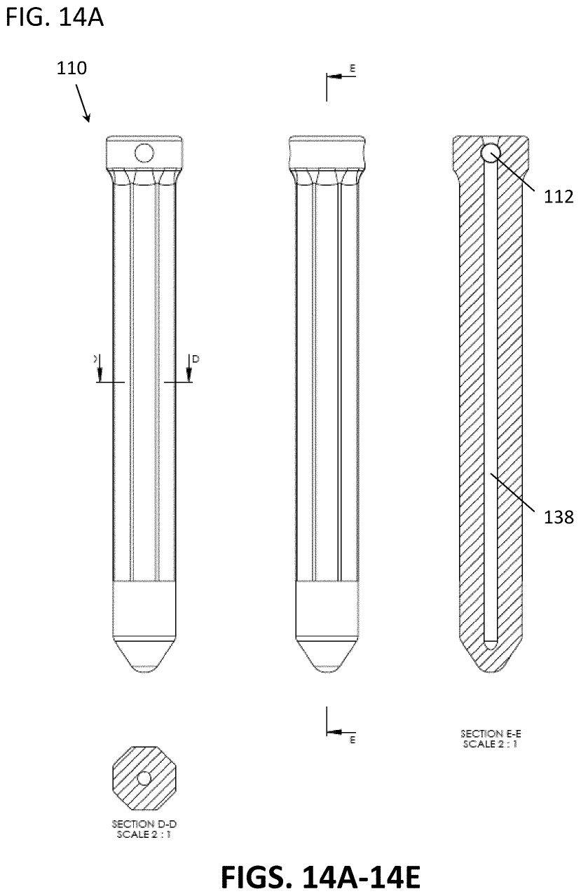

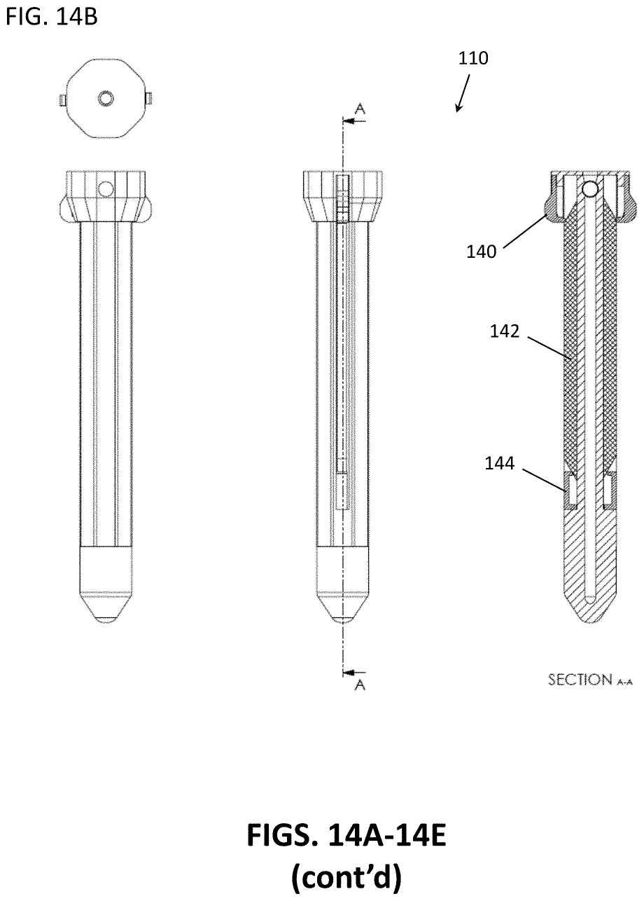

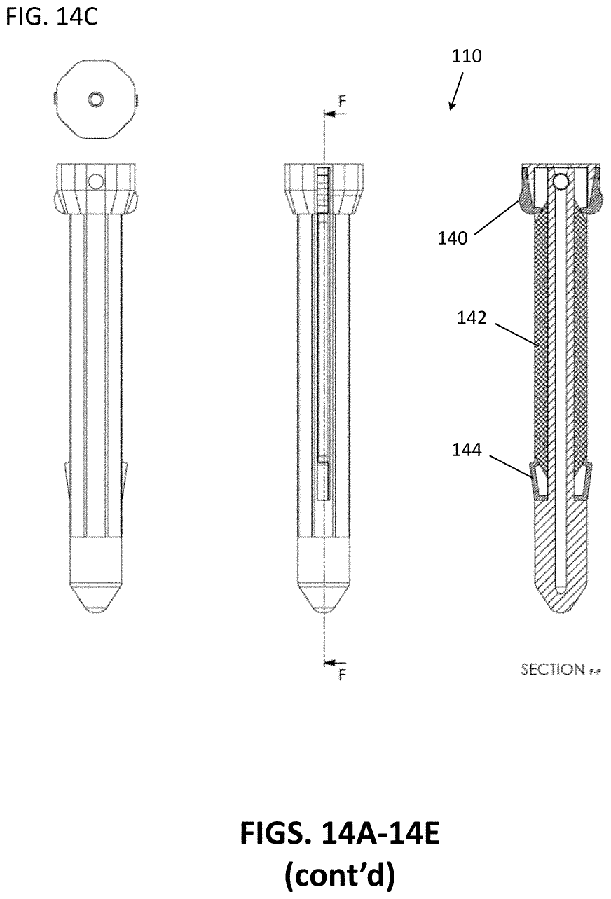

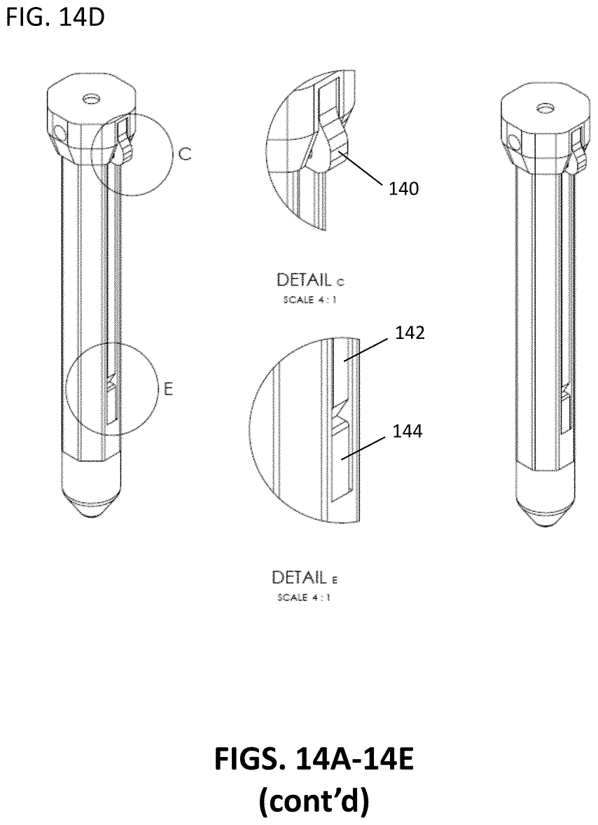

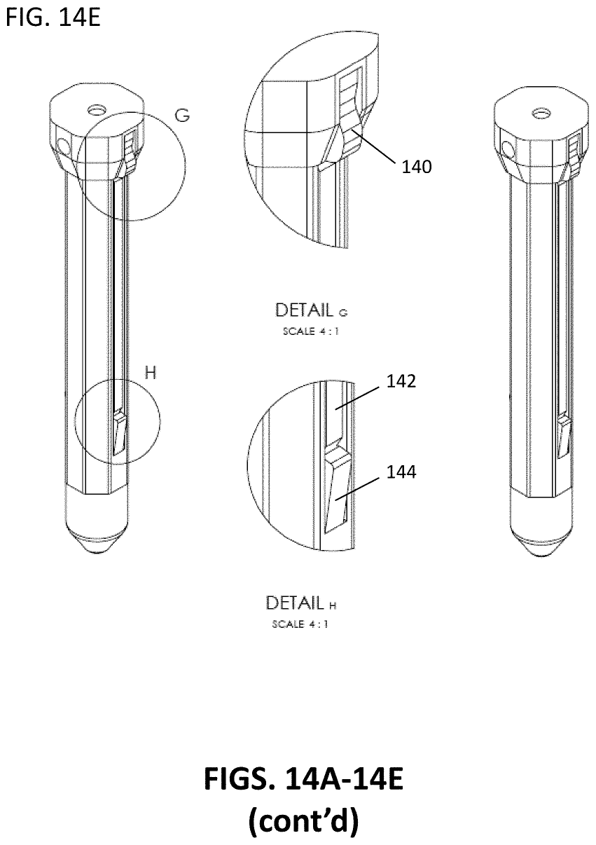

In other embodiments (not shown), top segment 126 can have no lip or flange at all, but can simply be a straight tube (e.g., similar to the axial segment depicted in FIG. 10). This embodiment allows cannula 120 to be inserted flush with or through a cranial opening. This can allow a smaller opening for a craniotomy, for example. The straight tube can terminate in an edge that is either substantially perpendicular to the axis of cannula 120, or the edge can be oriented at an angle, similar to embodiment 126B. In straight tube embodiments of top segment 126, obturator 110 can be used to grip and manipulate cannula 120, using frictional features for example (see, e.g., FIG. 14).

The dimensions of each of segments 122, 124, and 126 can be selected based on the parameters for use. For example, the overall diameter of the segments (corresponding to the overall diameter of the cannula) can be in the range of approximately 10-50 mm. An example length of base segment 122 can be approximately 40 mm diameter, and an example dimension of top segment 126 can be approximately 15 mm. In some embodiments, extension segments 124 can be from about 5 mm, about 10 mm, about 15 mm, about 20 mm, about 25 mm, about 30 mm, about 35 mm, about 40 mm, about 45 mm, and about 50 mm in diameter. Other configurations are also possible, as would be recognized by one of ordinary skill in the art based on the present disclosure.

FIGS. 10A-10B show details of an example modular connection mechanism for the plurality of axial segments. In these embodiments, a distal region of extension component 124 can have an outer diameter that is approximately the same as an inner diameter of a proximal region of a mating part (e.g., a base segment 122 or another extension section 124). Additionally, or alternatively, a proximal region of extension component 124 can have an outer diameter that is approximately the same as an inner diameter of a distal region of a mating part (e.g., a base segment 122 or another extension section 124). In other words, extension segment 124 can be inserted into base section 122. Likewise, a second extension segment 124 can be inserted into a first extension segment. The distal or proximal region of extension segment 124 can also have one or more external locking features 134, which connect to a corresponding internal locking feature 136 of the mating part. For example, external locking feature 134 can comprise one or more protruding pegs, and internal locking feature 136 can comprise a slotted pathway (e.g., Z-shaped pathway, a helical pathway, etc.) into which the peg is inserted (FIG. 10A). In other embodiments, external locking feature 134 can comprise a curved or hooked portion that wraps around internal locking feature 136, which can be a protruding peg (FIG. 10B). The overall wall thickness of the segments are designed such that the innermost and outermost surfaces of each part form a continuous surface when joined together (e.g., as seen in FIG. 1A). A top segment 126 can also have the same distal diameter and external/internal locking features as shown in FIGS. 10A-10B, for connection to an extension segment 124 or a base segment 122. In some embodiments, top segment 126 comprises little to no rim or lip around its most proximal portion (e.g., a low-profile design or “rimless”), as shown in FIG. 10B (upper panels), and in other embodiments, top segment 126 comprises a rim or lip around its most proximal portion, as shown in FIG. 10B (lower panels).

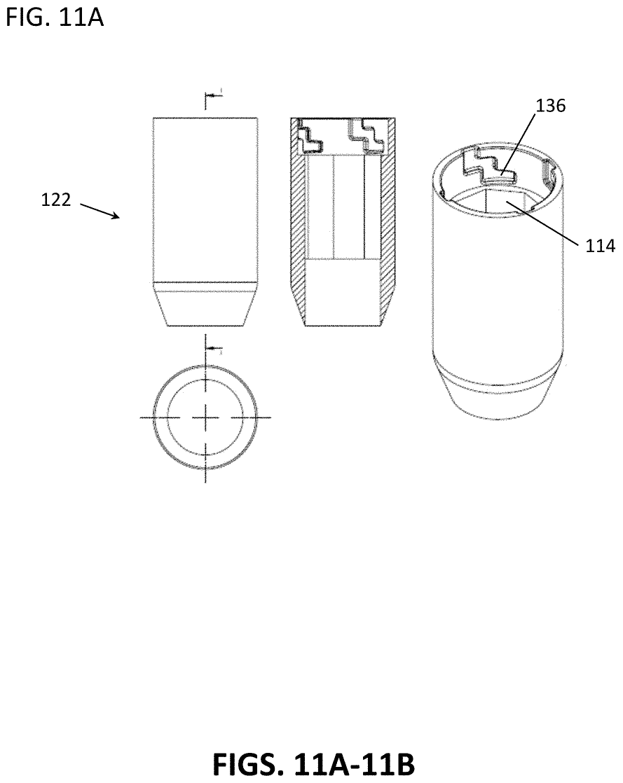

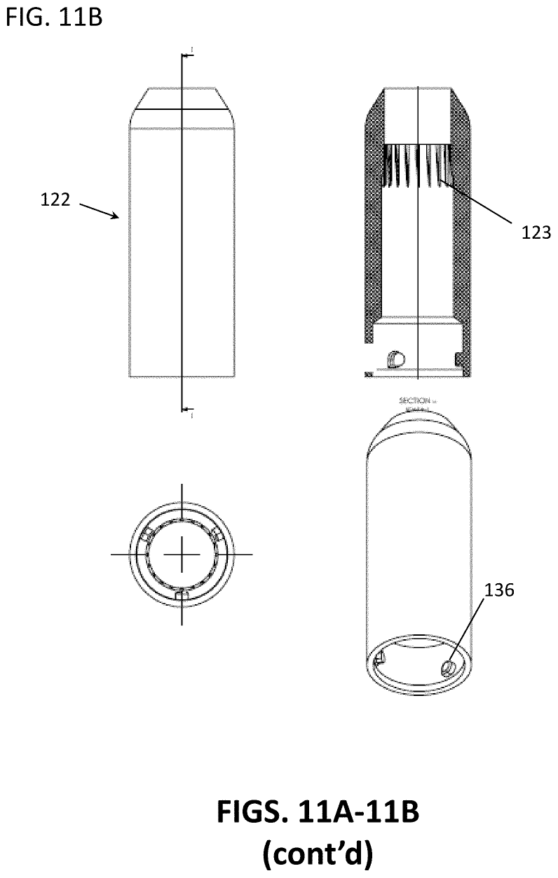

FIGS. 11A-11B include representative views of various distal base segments 122, including embodiments comprising internal and external locking features, as described above with respect to FIGS. 10A-10B. Additionally, FIG. 11A includes a distal base segment 122 comprising a rotational locating feature 114 having at least one inner planar surface that interfaces with a corresponding external planar surface of an obturator to prevent rotation of the obturator within the cannula. FIG. 11B includes a distal base segment 122 comprising a rotational locating feature 114 comprising a plurality of splines 123 on the inner surface of the cannula 120 that interface with a corresponding plurality of splines 123 on the outer surface of the obturator 110 to prevent rotation of the obturator within the cannula (see, e.g., FIG. 2).

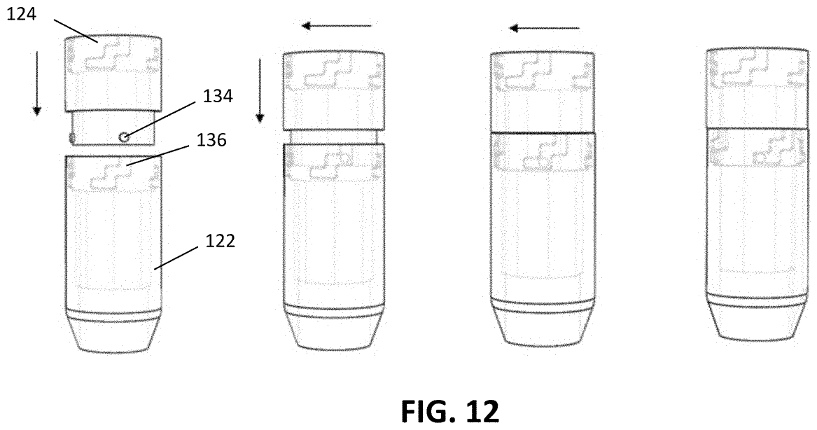

FIG. 12 depicts an example method of joining a base segment 122 and an extension segment 124, illustrated in a series of steps. To join the segments, a user would align internal and external locking features 134 and 136, twisting lightly and pushing the segments together. To remove the segments, the reverse action is performed. In some embodiments, a key slot tightly secures the components together so that there is no additional movement once attached. It is to be noted that, although the locking mechanism is described with reference to a key slot, other locking mechanisms can be substituted without departing from the scope of the invention, as would be recognized by one of ordinary skill in the art based on the present disclosure. In some embodiments, the key slot is not perfectly circular, but may comprise a flat face that increases resistance required to disassemble the components of the cannula. Additionally, FIG. 12 illustrates that the distal end of the cannula can comprise a tapered end, which can improve transitioning with the obturator.

Additionally, when the plurality of axial segments are assembled, the rotational locating features 114 also align, allowing the user to slide the obturator unimpeded within the cannula to correctly position the atraumatic tip independent of the cannula length. This provides a physical feedback to the operator that the device is properly assembled and also allows the surgeon to manipulate the obturator like a stylet. If desired, the surgeon can twist and position the cannula by manipulating only the obturator, since the obturator does not rotate within the cannula.

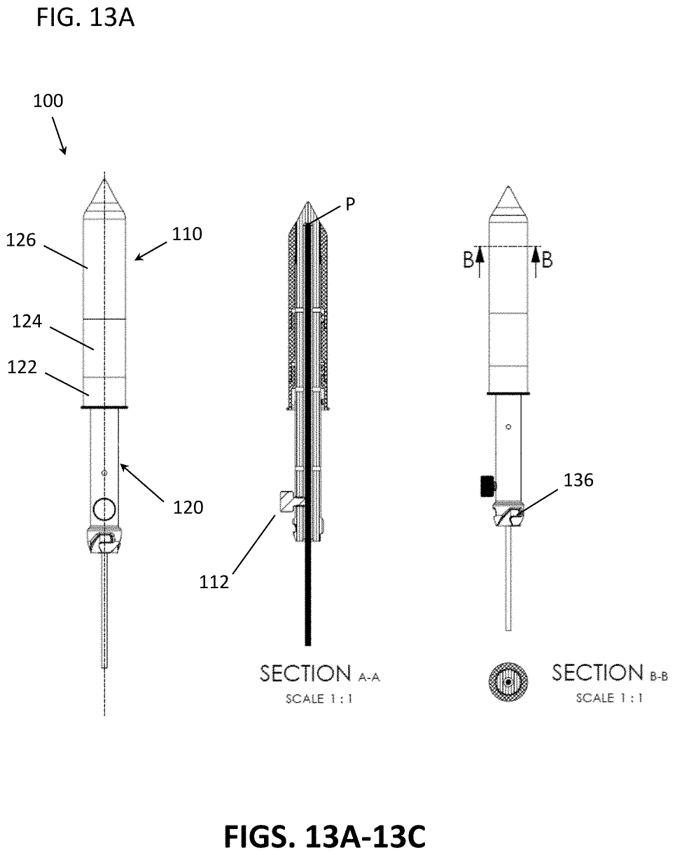

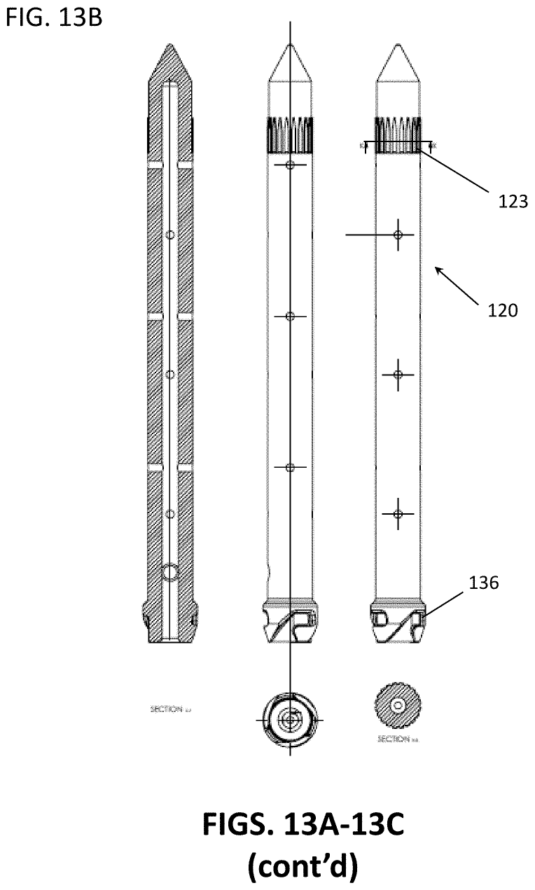

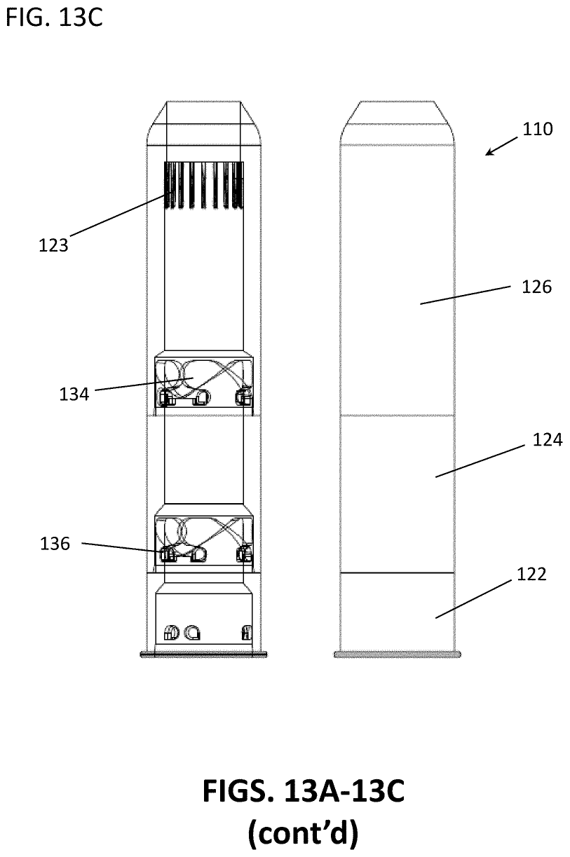

FIGS. 13A-13C include various cross-sectional views of a retractor system 100 comprising an obturator 120 and a cannula 110, each comprising the features described above. Additionally, as shown in FIG. 13A, obturator 120 comprises a substantially cylindrical shape, but is not cross-sectionally octagonal. In some embodiments, highlighted in FIG. 13B, the system includes an internal locking feature 136 as part of the proximal end of the obturator 120 for interfacing with a corresponding external locking feature 134 in the proximal end of the cannula 120 to facilitate removal of the cannula 120 using the obturator 110 (see, e.g., FIG. 7). In some embodiments, as highlighted in FIGS. 13B-13C, the system includes a distal base segment 122 comprising a rotational locating feature 114 comprising a plurality of splines 123 on the inner surface of the cannula 120 that interface with a corresponding plurality of splines 123 on the outer surface of the obturator 110 to prevent rotation of the obturator within the cannula (see, e.g., Section B-B in FIG. 13A). Additionally, as highlighted in FIG. 13C, the system includes a cannula 120 that includes an axial extension segment joining the proximal top segment to the distal base segment using one or more an external locking features 134 comprising a curved or hooked portion that wraps around internal locking feature 136, which can be a protruding peg (see, e.g., FIG. 10B).

FIGS. 14A-14E illustrate various embodiments of obturator 110. In one embodiment shown in FIG. 14A, obturator 110 can be a substantially solid component with a passageway 138 for housing a guidance probe. The length of the passageway can be selected to operate with the design parameters of the selected probe. For example, in some embodiments the probe can be advanced within the obturator to 5 mm from the tip. The probe can be secured in place using a retention element 112. In the example embodiments shown, the top of the obturator has a shaft collar mechanism, and the probe is pinned in place by twisting a set screw. The distal end of obturator 110 forms a continuous outer surface with cannula 120 at the distal end of retractor 100, and it is used as the working end for dilation or retraction. Obturator 110 slides freely within cannula 120 so that it can be easily removed once cannula 120 is positioned.

Referring to FIGS. 14B-E, another embodiment of obturator 110 is shown. FIGS. 14B and 14C are various elevation views, while FIGS. 14D and 14E are perspective views. In this example, obturator 110 has additional features that allow obturator 110 to grip onto cannula 120. A user can depress buttons 140 at the top of obturator 110, translating an intermediate piece 142, which causes the distal flanges 144 to deflect outwards. Flanges 144 frictionally engage with the inside of cannula 120 so that the user can lift cannula 120 out of the tissue. FIGS. 14B and 14D depict flanges 144 in a retracted position, and FIGS. 14C and 14E show flanges 144 in an extended position.

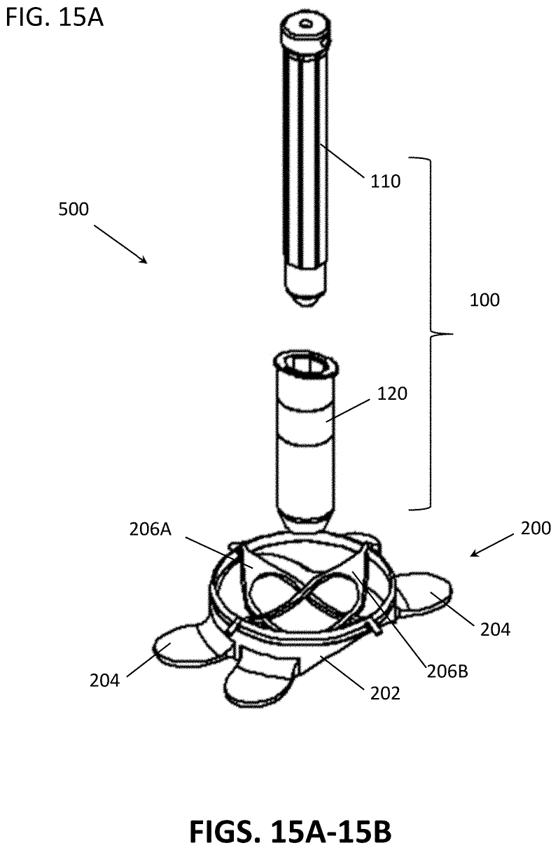

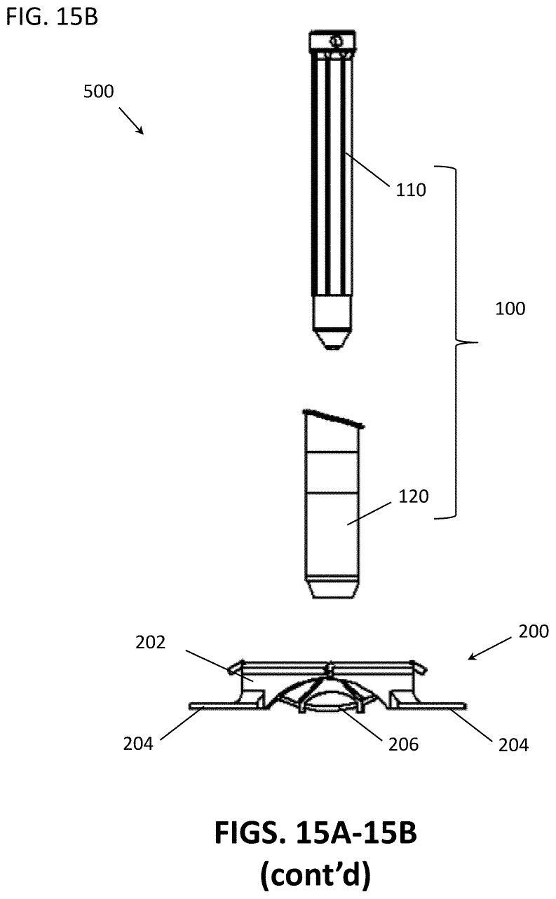

Another aspect of the present disclosure provides a retractor system 500 comprising a retractor 100 and an adjustable mount or fixation device 200 configured to position a medical tool (e.g., a cannula) in one or more stable positions. FIGS. 15A and 15B are exploded perspective and elevation views, respectively, of an example embodiment of a retractor system 500. Adjustable mount 200 includes a base 202, legs or tabs 204, and an adjustable cannula attachment portion 206, at least part of which is recessed within the base 202. Adjustable mount 200 can be placed around the entry point of cannula 120 and used to adjust, hold, and lock cannula 120 in place at the chosen angle. Tabs 204 can be placed on the skin or skull of a subject. In some embodiments, a surgeon can position an adjustable mount 200 on a patient by sliding tabs 204 between the scalp and skin, using tension from the skin against the rigid surface of the mount to holds the tabs in place. Alternately, tabs 204 can optionally be equipped with attachment and/or mounting features and can thus position the adjustable mount 200 by screwing into the skull, stapling to the scalp, adhesion (e.g., Ioban, tegaderm), or other attachment mechanisms.

Adjustable mount 200 additionally can include an adjustable cannula attachment portion 206, configured to be initially rotated and translated around base 202 and then locked into place once the desired location and direction for cannula 120 have been determined. The cannula attachment portion 206 is at least partially recessed in adjustable mount 200 such that cannula 120 can be as close to the brain as possible. This reduces the number of segments needed, as well as increasing the working area for the practitioners.

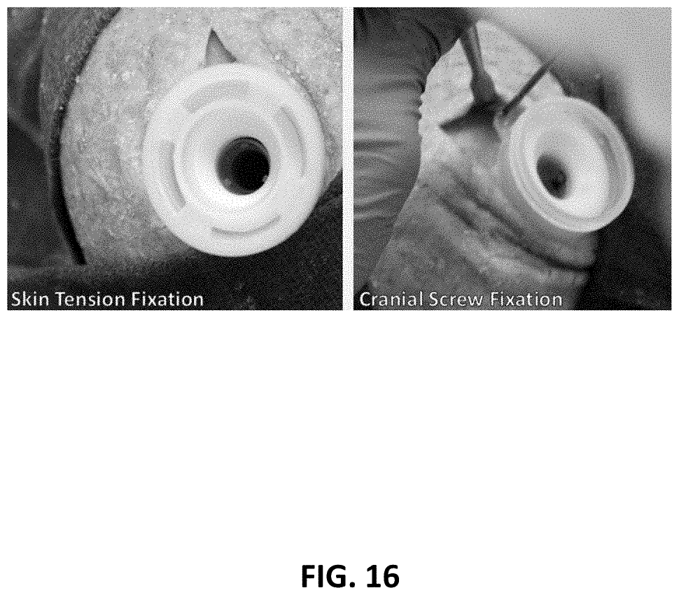

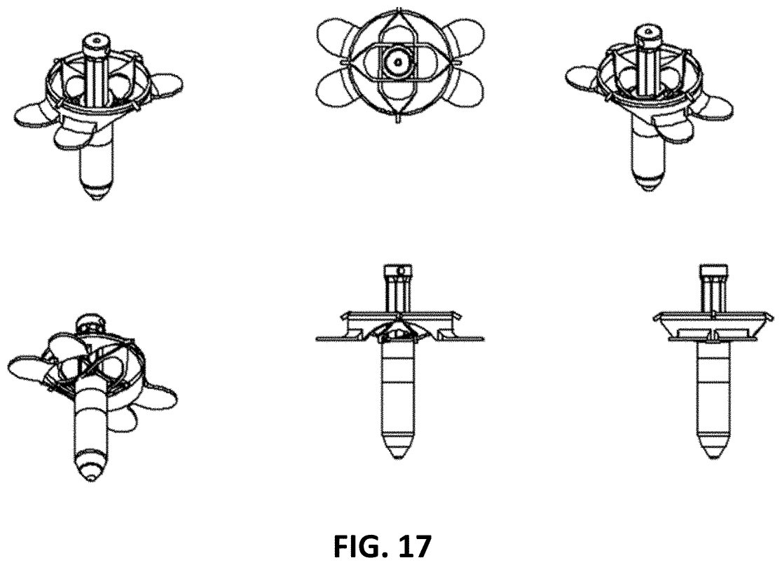

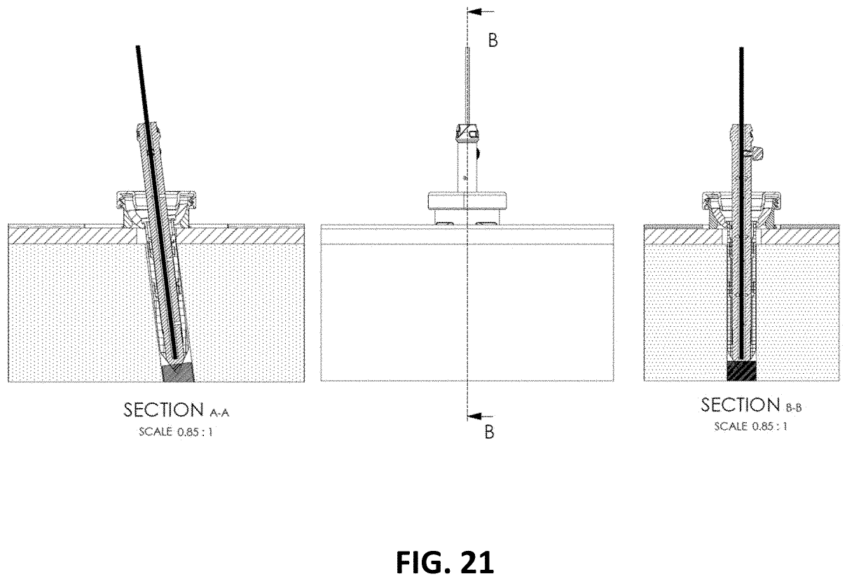

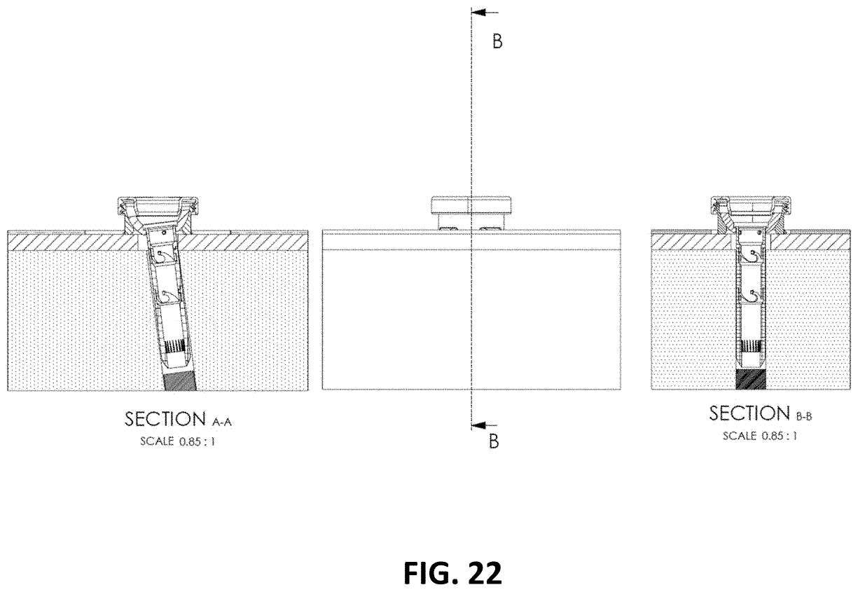

FIG. 16 includes images of an example adjustable mount 200 in a surgical application. In some embodiments, cannula attachment portion 206 is configured such that retractor system 100 can be translationally positioned substantially anywhere within base 202. Retractor system 100 can be axially rotated 360° within cannula attachment portion 206, and the angular positioning of retractor 100 with respect to a normal entry plane (e.g., see FIGS. 21-22) can be at least ±20°. In some embodiments, orthogonal sliders (e.g., 206A, 206B) can used for this positioning by capturing cannula 120, and, once the position is reached, the sliders are locked into place by twisting a cap on base 202. The sliders can be formed with a concave shape that puts the proximal surface of cannula 120 substantially flush with the surface of the brain. FIGS. 17-19 includes representative views of a retraction system 100 comprising an adjustable mount 200 with cannula 120 and/or obturator 110 inserted into the mount.

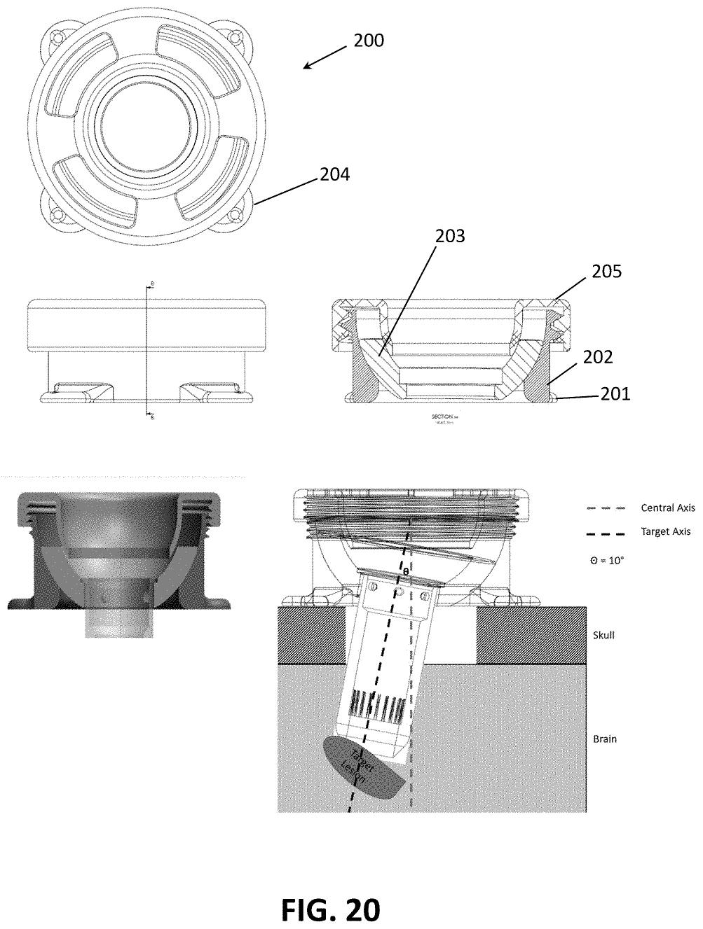

In some embodiments, as shown in FIGS. 20-22, a retractor system 100 can include an adjustable mount 200 that includes a base 202 for securing the mount 200 to a subject, and an adjustable medical tool attachment portion 203 that is at least partially recessed in the base 202, and a tension cap 205 configured to apply varied amounts of pressure to the medical tool attachment portion to secure the tool in one or more stable positions. The base 202 can comprise at least two opposing base tabs 204 and/or a concentric lip 201 at the bottom of the mount 200 to secure the base to the subject. In some embodiments, the base tabs 204 and/or the concentric lip 201 facilitate attachment to the subject without the need for additional attachment means. In some embodiments, the base tabs 204 are perforated for insertion of an additional attachment means (e.g., screws) to secure the mount 200 to the subject.

In some embodiments, the medical tool attachment portion 203 is generally concave in shape with respect to the tension cap 205, such that the apex portion of the medical tool attachment portion 203 is at least partially recessed within the base 202 (FIG. 20). When a desired position for the medical tool is reached, the medical tool attachment portion 203 is held in a substantially stable position using the tension cap 205. For example, the tension cap 205 can be detachably coupled to the base 202 with a screw interface, such that as the tension cap 205 is tightened to the base 202, increased force or tension is applied to the medical tool attachment portion 203, thereby securing the medical tool in place. In some aspects, the configuration of the base, 202, the medical tool attachment portion 203, and the tension cap 205 resemble a ball joint, which allows for 360° of movement (and up to 20° from the y-axis). The tension applied using the tension cap 205 is adjustable, and the amount of tensions applied will depend on various factors such the type of medical tools being used, the nature and location of the target lesion, and the like.

Securing the medical tool in one or more stable positions includes securing the tool at an angle with reference to a surface-to-target location, such as depicted in FIGS. 20-22. For example, FIG. 21 includes a cross-sectional view of an adjustable mount 200 with a cannula 120 and obturator 110 inserted into it at an angle, with respect to a normal entry plane (e.g., with reference to a surface-to-target location; section B in FIGS. 21-22). This angle can be about ±20° or less. Additionally, FIG. 22 includes a cross-sectional view with the obturator 110 removed, thus providing an open working channel for one or more medical tools to treat the target tissue.

As would be recognized by one of ordinary skill in the art based on the present disclosure, any suitable medical tools can be used with the retractor system 100 and adjustable mount 200, including but not limited to, a cannula, bipolar forceps, dissectors, biopsy forceps, a suction device, a camera light source, a guidance probe, coagulation forceps, micro-instruments, and the like. Additionally, it would be recognized that the adjustable mount 200 can be used independently of the retractor system 100, and on any type of tissue of a subject that is to be retracted to access a target tissue, including but not limited to, neural tissue, cerebral ventricles, cerebral parenchyma, spine and other solid organs such as liver, spleen or kidney. Further, the devices and systems of the present disclosure can be used to treat any lesion in a target tissue, including but not limited to, a cystic lesion, an infection (e.g., cerebral abscess), a primary or metastatic tumor, an intracerebral or intraventricular hemorrhage, an intracranial hematoma, a vascular abnormality or malformation, an intraventricular cyst, and the like.

### 3. Methods of Use

Embodiments of the present disclosure also include a method of performing a medical procedure using the systems and devices described herein. In some embodiments, the procedure is performed to gain access to or visualize a surgical field (e.g., target tissue). In accordance with these embodiments, the method comprises inserting the cannula and obturator into the tissue adjacent to a target lesion using an guidance probe, and adjusting the length of the cannula by adding or removing at least one axial segment. In some embodiments, the method is performed prior to and/or during a surgical procedure, wherein the cannula is not fully removed until completion of the surgical procedure. In some embodiments, inserting the cannula and obturator comprises use of an adjustable cannula mounting device, wherein the cannula and obturator are inserted at an angle with reference to a direct target lesion-to-scalp axis.

Another aspect of the present disclosure provides a method of performing brain retraction using the disclosed brain retraction devices and systems. In some embodiments, a preparatory step or pre-operative planning is performed. This can include magnetic resonance imaging (MRI) and/or computed tomography (CT) scanning of the region to be retracted. This can assist the surgeon in calculating the depth and trajectory of surgery, thereby allowing the surgeon to select the number and type of extension pieces to assemble for the cannula. Next, a craniotomy can be performed in conjunction with the method. In some embodiments, one scalp incision is made to allow a burr hole placement or craniotomy, which can be slightly larger than the diameter of tube to facilitate a few degrees of freedom and/or motion.

If an adjustable mounting device/system (e.g., 200) is used, the device is positioned over the surgery site. The adjustable mounting device can be fixed in place using a number of methods, including connection hardware (e.g., screws, staples, etc.), adhesion to the skin surface, or capturing the tabs inside the scalp. Alternately, a cannula can be used without the adjustable mounting device by selecting a top segment of the cannula that interfaces with other types of surgical equipment, such as a halo retractor system.

Next, an imaging probe can be inserted into the obturator (e.g., 110) and fixed in place with a retention element (e.g., 112). The obturator is then assembled into the cannula to form a complete retractor system. The retractor system can then be optionally coupled to an adjustable mounting device, as described further herein.

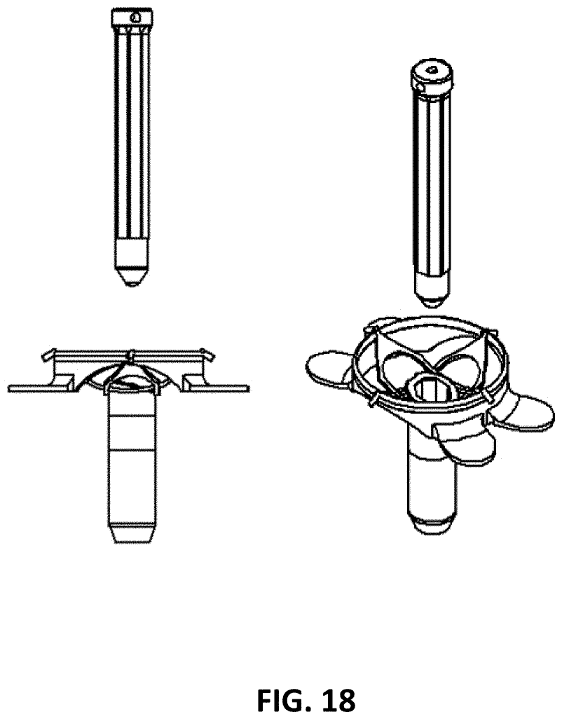

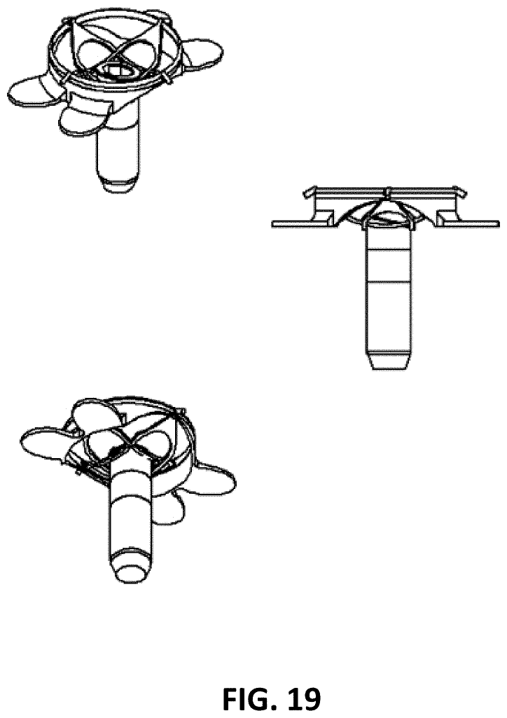

The assembled retractor system can then be inserted into the brain between the selected sulci until the desired depth is reached, using the probe to navigate. The angle of entry can be adjusted during insertion by moving the adjustable holders of the adjustable mounting device, thus widening the field of view, if in use. The holding portion of the adjustable mounting device can be initially unlocked and then locked into place once the insertion is complete. Once the retractor device is fixed in position, the obturator is removed (FIG. 18), and surgery can begin. FIG. 19 depicts an example embodiment of a cannula and an adjustable mounting device in a surgical configuration.

In some embodiments, if additional length is required during insertion or mid-surgery, the top segment (e.g., 126) of the obturator can be temporarily removed, and an additional extension segment (e.g., 124) can be added to the cannula. In other embodiments, if additional length is required, a surgeon may first choose a distal base cannula segment of a desired length, and then add an addition axial segment or the top segment upon assessment of the target tissue, without first removing a segment. Extending cannula length may be necessary, for example, if accessing a lesion which extends away from the cannula deeper into the brain. It is also possible to remove an extension segment and replace it with an extension segment of a different length (e.g., during a medical procedure). Once the additional and/or replacement segments are reassembled, the obturator is reintroduced into the cannula, and insertion is continued to the desired depth. The obturator is then again removed from the cannula and surgery can resume. Hence, it is possible to reconfigure nearly all the segments of the cannula, leaving the base segment in place during a retraction. If angle adjustment is required mid-surgery, the adjustable mounting device can be unlocked to allow the holding portion to be adjusted. The device is then reoriented to the desired angle and locked into the new position.

Another aspect of the present disclosure provides all that is described and illustrated herein. Although the devices and methods disclosed herein are directed toward brain retraction, it will be evident to a person of skill in the art that these devices and methods can also be useful in other types of surgical procedures where cannulas are used. Some non-limiting examples include use in other solid organs and in laparoscopic surgery.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A tissue retraction system comprising:
an obturator comprising a housing for one or more surgical tools; and
a cannula configured to accept the obturator and comprising a plurality of axial segments, wherein the plurality of axial segments comprises at least one distal base segment and at least one proximal top segment;
wherein the plurality of axial segments are modularly stackable and configured to be added and/or removed prior to and/or during a medical procedure without removing the cannula from the tissue.

2. The system of claim 1, wherein the plurality of axial segments of the cannula comprises one or more extension segments disposed between the distal base segment and the proximal top segment.

3. The system of claim 1, wherein the proximal top segment comprises an extension tab for interfacing with an external retractor tool.

4. The system of claim 1, wherein the surgical tool is one or more of an aspiration canula, a micro-dissector, a micro-scissors, a bipolar electrocoagulation image guidance probe, an ablation tool, a tissue removal tool, or a combination thereof.

5. The system of claim 1, wherein the obturator comprises a retention element to secure the one or more surgical tools in position.

6. The system of claim 1, wherein at least one area along the longitudinal axis of the obturator is planar and interfaces with at least one corresponding area along the longitudinal axis of the cannula to prevent rotation of the obturator within the cannula.

7. The system of claim 1, wherein the distal base segment of the cannula comprises a plurality of splines on its inner surface that interface with a corresponding plurality of splines on the outer surface of the obturator to prevent rotation of the obturator within the cannula.

8. The system of claim 1, wherein the plurality of axial segments of the cannula comprise an external locking feature and/or an internal locking feature, such that each segment can be removably coupled to a separate segment.

9. The system of claim 1, wherein the plurality of axial segments of the cannula are from about 5 mm to about 50 mm in height.

10. The system of claim 1, wherein the plurality of axial segments of the cannula are from about 5 mm to about 50 mm in diameter.

11. The system of claim 1, wherein the obturator comprises two proximally positioned buttons functionally coupled to two distally located flanges, such that depressing the buttons causes the flanges to engage the cannula, thereby facilitating removal of the cannula upon removal of the obturator.

12. The system of claim 1, wherein the proximal end of the obturator comprises an external locking feature or an internal locking feature that engages a corresponding internal or external locking feature in the proximal end of the cannula, such that engagement of the proximal end of the obturator with the proximal end of the cannula facilitates removal of the cannula using the obturator.

13. The system of claim 1, wherein the tissue is neural tissue.

14. The system of claim 1, further comprising an adjustable mount configured to position the cannula in one or more stable positions.

15. The system of claim 14, wherein the adjustable mount comprises:
a base for securing the mount under the scalp of a subject;
an adjustable cannula attachment portion comprising an opening for insertion of the cannula, wherein at least a portion of the adjustable cannula attachment portion is recessed in the base; and
a tension cap configured to apply varied amounts of pressure to the cannula attachment portion to secure the cannula in one or more stable positions.

16. The system of claim 14, wherein the base comprises at least two opposing base tabs and/or a concentric lip at the bottom of the mount.

17. The system of claim 14, wherein the adjustable cannula attachment portion has a concave shape with respect to the tension cap.

18. An adjustable mounting device for at least one medical tool, the device comprising:
a base for securing the mount to a subject;
an adjustable medical tool attachment portion comprising an opening for insertion of at least one medical tool, wherein at least a portion of the adjustable medical tool attachment portion is recessed in the base; and
a tension cap configured to apply varied amounts of pressure to the medical tool attachment portion to secure the tool in one or more stable positions.

19. The device of claim 18, wherein the base comprises at least two opposing base tabs and/or a concentric lip at the bottom of the mount.

20. The device of claim 19, wherein the base tabs and/or the concentric lip facilitate attachment to the subject without the need for additional attachment means.

21. The device of claim 19, wherein the base tabs are perforated for insertion of an additional attachment means to the subject.

22. The device of claim 18, wherein the adjustable medical tool attachment portion has a concave shape with respect to the tension cap.

23. The device of claim 18, wherein the at least one medical tool comprises a cannula, bipolar forceps, dissectors, biopsy forceps, a suction device, a camera light source, a guidance probe, coagulation forceps, and/or a micro-instrument.

24. The device of claim 18, wherein the base secures the mount to the subject's skull.

25. The device of claim 18, wherein securing the medical tool in one or more stable positions comprises securing the tool at an angle with reference to a surface-to-target location.

26. The device of claim 25, wherein the angle is about 20 degrees or less.

27. A method of performing a medical procedure using the system of claim 1, the method comprising:
inserting the cannula and obturator into the tissue adjacent to a target lesion using an guidance probe; and
adjusting the length of the cannula by adding or removing at least one axial segment.

28. The method of claim 27, wherein the method is performed prior to and/or during a surgical procedure, wherein the cannula is not fully removed until completion of the surgical procedure.

29. The method of claim 27, wherein inserting the cannula and obturator comprises use of an adjustable cannula mounting device, wherein the cannula and obturator are inserted at an angle with reference to a direct target lesion-to-scalp axis.
